(19) United States
(12) Patent Application Publication
(10) Pub. No.: US 2009/0326287 A1
(43) Pub. Date: Dec. 31, 2009

(54) METHOD FOR PRODUCING O-XYLENE

(75) Inventors: Götz-Peter Schindler, Ludwigshafen (DE); Thomas Heidemann, Viernheim (DE); Christian Miller, Deidesheim (DE); Godwind Tafara Peter Mabande, Mannheim (DE); Bianca Stäck, Mannheim (DE); Thomas Hill, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/307,135

(22) PCT Filed: Jul. 3, 2007
(86) PCT No.: PCT/EP07/56679

§ 371 Date: Dec. 31, 2008

(30) Foreign Application Priority Data
Jul. 3, 2006 (EP) 06116501.5

(51) Int. Cl. C07C 15/08; C07C 6/00; C07C 5/00

(52) U.S. Cl. 585/251; 585/322; 422/187

(57) ABSTRACT

A process for preparing o-xylene comprises the steps of
a) dimerizing 2-butenes to 3,4- and/or 2,3-dimethylhexenes and
b) aromatizing the 3,4- and/or 2,3-dimethylhexenes under dehydrogenating conditions to give o-xylene
   and is suitable for the selective preparation of o-xylene.

The invention relates to a process for preparing o-xylene by dimerizing butenes and aromatizing the resulting product under dehydrogenating conditions.

o-Xylene (1,2-dimethylbenzene) is used in industrial scale amounts, for example, for the synthesis of phthalic anhydride whose uses include the preparation of plasticizers.

Nowadays, it is usually prepared on the industrial scale from natural mixtures, obtained from coal tar and mineral oil, of the three isomeric xylenes (o-, m- and p-xylene) by comparatively complicated separation processes, such as fractional crystallization or absorption with molecular sieves. Owing to the limited natural occurrence of o-xylene and the complicated removal of other xylene isomers, there is the constant task of providing novel efficient processes for preparing o-xylene which are suitable especially for industrial scale use.

It is known that aromatic hydrocarbons can be obtained by catalytic dehydrogenating aromatization of open-chain hydrocarbons (see, for example, Catalysis VI, p. 535-542, ed. by P. H. Emmet, Reinhold Publishing Co., New York, 1958).

U.S. Pat. No. 3,449,461 describes the dehydrogenating aromatization of open-chain C₆ to C₂₀ paraffins to aromatic hydrocarbons including o-xylene with the aid of a sulfur catalyst which comprises a noble metal such as palladium or platinum

US-A 2004/0044261 describes a process for selectively preparing p-xylene by converting C₈ isoalkenes or alkenes over a catalyst which comprises a molecular sieve laden with a noble metal of transition group VIII.

DE-A 197 27 021 describes a process for preparing C₈ aromatics from butenes by dehydrogenating olefinically unsaturated C₈ hydrocarbon mixtures obtainable by dimerizing technical C₄ cuts over a catalyst which comprises at least one element of the platinum group on an amphoteric ceramic support. The main reaction product is ethylbenzene; in addition, o-xylene is also formed.

It has now been found that o-xylene can be prepared selectively by dimerizing 2-butenes to 3,4- and 2,3-dimethylhexenes with subsequent dehydrocyclization.

A selective dehydrogenating aromatization of 2,3-dimethylhexane is described by V. I. Komarewsky and W. C. Shand in J. Am. Chem. Soc. 66 (1944) 1118. Starting from 2-butenes, thought the selective preparation of o-xylene is to date unknown.

The invention therefore provides a process for preparing o-xylene, comprising the steps of
a) dimerizing 2-butenes to 3,4- and/or 2,3-dimethylhexenes and
b) aromatizing the 3,4- and/or 2,3-dimethylhexenes under dehydrogenating conditions to give o-xylene.

The process according to the invention enables technically simple preparation of o-xylene in good yields and with high selectivity.

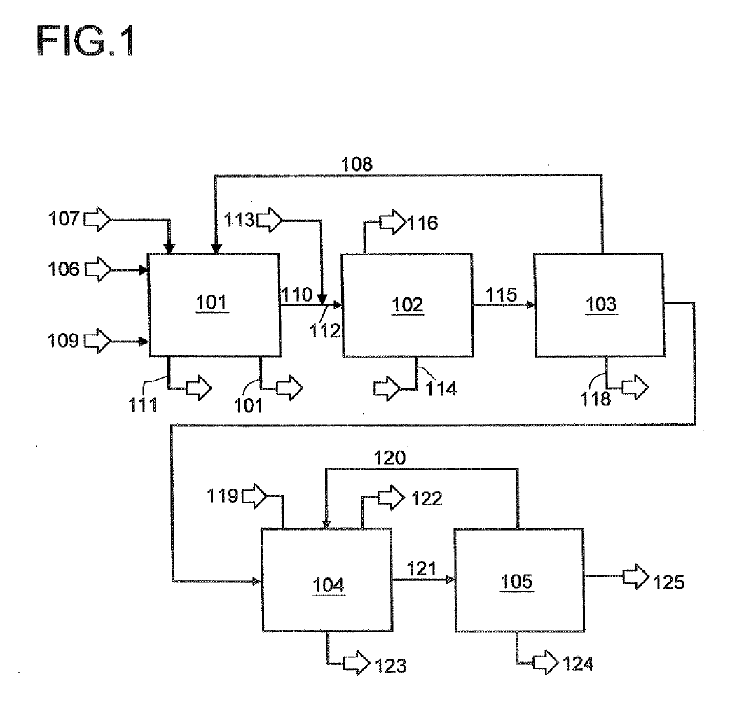

FIG. 1 shows a schematic of an apparatus for performing the process according to the invention starting from n-butane or raffinate II or III.

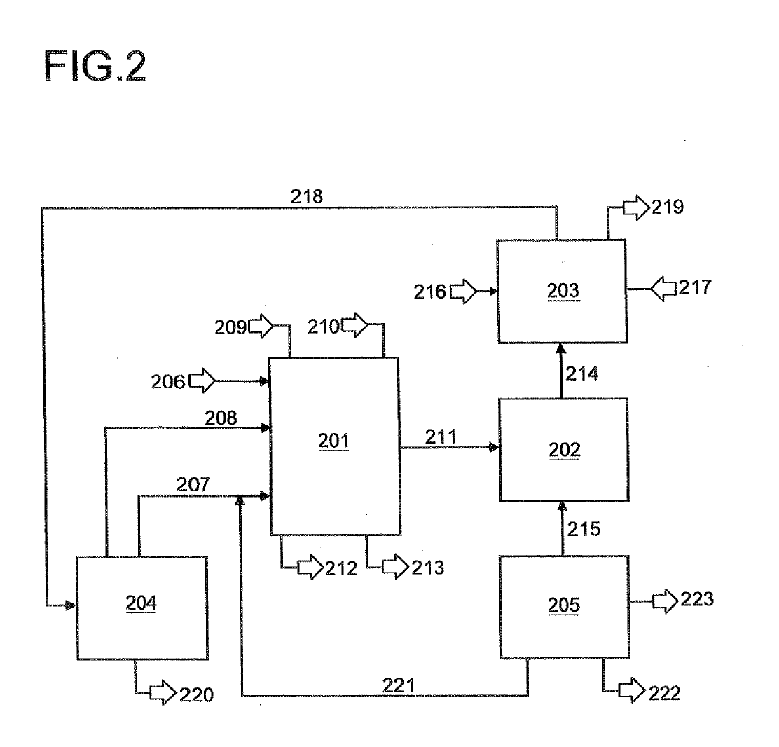

FIG. 2 shows a schematic of an apparatus for performing a preferred variant of the process according to the invention starting from n-butane or raffinate II or III, in which a butane dehydrogenation and the dehydrogenating aromatization are performed in one reactor.

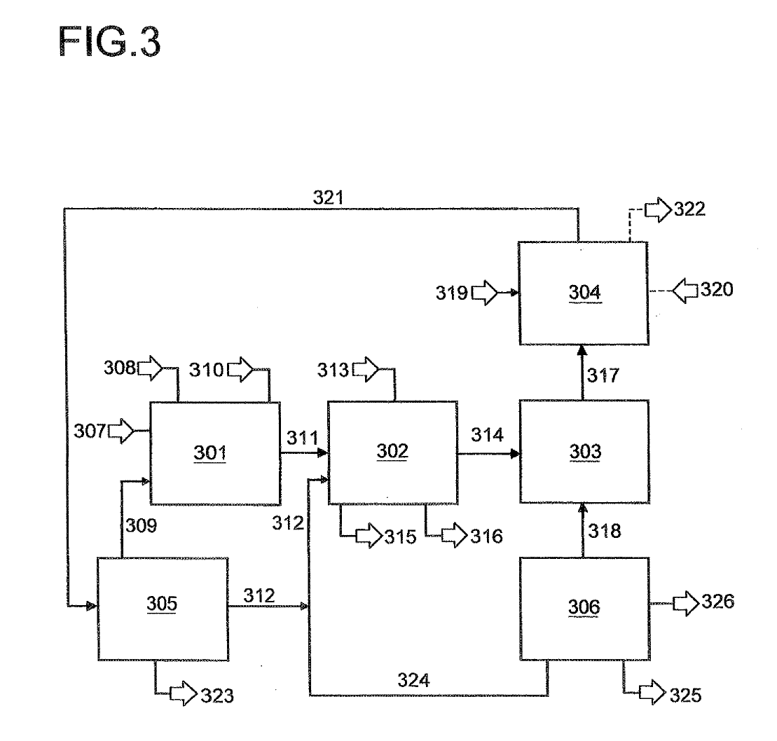

FIG. 3 shows a schematic of an apparatus for performing a further preferred variant of the process according to the invention starting from n-butane or raffinate II or III, in which a butane dehydrogenation and the dehydrogenating aromatization are connected in series.

### DIMERIZATION/STAGE (A)

The task of the first stage (a) is to convert the 2-butenes present in the feed stream to 3,4- and/or 2,3-dimethylhexenes with maximum conversion and selectivity.

The dimerization of n-butenes to 3,4- and/or 2,3-dimethylhexenes is possible by means of heterogeneous and also homogeneous catalysis, preference being given to heterogeneous processes.

Suitable catalysts are those which have a high selectivity for the formation of 3,4- and/or 2,3-dimethylhexenes.

Examples of such catalysts are:
a) ion exchangers based on synthetic resins, such as Amberlyst®.
Such catalysts are described, for example, in U.S. Pat. No. 4,463,211.
b) Alumosilicates, for example as described in GB-A 1,116,474.
c) Phosphoric acid, especially applied to a support material such as silica.
Such catalysts are described, for example, in Erdöl und Kohle, 1959, 549.
d) Sulfates of elements of transition group VIII of the Periodic Table of the Elements, preferably of Fe and Co, these sulfates preferably being applied to an aluminum oxide support.
Such catalysts are described, for example, in U.S. Pat. No. 4,423,267.
e) Sulfate and/or tungstate-modified oxides of Al, Al/Si, Ti and Zr.
Such catalysts are described, for example, in U.S. Pat. Nos. 5,113,034 and 5,883,036.

Preference is given to catalysts of group (e). Preferred oxides are aluminum oxides, aluminosilicates and titanium dioxide, more preferably aluminum oxides and aluminosilicates, especially aluminum oxides, such as γ-, θ- and δ-Al₂O₃.

A preferred modifying anion is sulfate (SO₄²⁻). The metal oxides used in accordance with the invention preferably also serve as a support material, but may also be applied to a support material such as silica gel.

The oxides may be used as such or be obtained from a precursor.

Suitable precursors are, for example, salt solutions, such as chlorides, oxychlorides and nitrates, especially of Ti and Zr. The salts are preferably water-soluble and form a hydroxide precipitate of the metal on addition of a base. Suitable bases comprise, for example, ammonium hydroxides and alkylammonium hydroxides, which are added to adjust the pH to about from 9 to 11 and bring about the precipitation of the metal as the hydroxide.

It is equally possible to use alkoxides of the metals mentioned, for example zirconium n-propoxide or titanium i-propoxide, which are then hydrolyzed with water to the corresponding hydroxides.

Also suitable are oxide hydrates, such as aluminum oxide hydrates and silicon aluminum hydroxide gels.

Any material which is capable of forming sulfates or tungstates on calcination with the metal oxides mentioned is suitable as the anion, i.e. sulfate or tungstate source. Examples include H₂S, SO₂, mercaptans, sulfur- and halogen-containing compounds such as fluorosulfonic acid, SOCl₂ and SO₂Cl₂ or mixtures thereof, and also ammonium metatungstate.

The anion can be combined with the oxide or its precursors by any processes, for example by immersion in or impregnation with H₂SO₄, preferably diluted with water, or ammonium sulfate solution, preferably aqueous solution, with subsequent drying at from 100 to 150° C.

After the anion source has been absorbed by the oxide or its precursor and if appropriate dried, a calcination is generally effected, if appropriate in an atmosphere which promotes the formation of the anion from an appropriate precursor, for example an oxidizing atmosphere such as air.

The calcination is generally performed at temperatures of from 350 to 650° C. for sulfates and from 350 to 800° C. for tungstates. Preference is given to a temperature of from about 450 to 550° C., in particular about 500° C.

The duration of calcination is generally from 0.5 to 30 h, preferably from 0.5 to 24 h, in particular from 0.5 to 10 h.

Alternatively, a precursor of the oxide, for example a hydroxide, can be calcined, preferably at temperatures of from 350 to 600° C., in order to perform the conversion to the oxide, and then admixed with the anion source as described above.

The concentration of the sulfate or tungstate is preferably from 1 to 20% by weight, preferably from 3 to 10% by weight, based on the weight of the metal oxide.

The catalyst used in accordance with the invention may further comprise a transition metal compound from the group of Fe, Co, Ni and Cr, preferably Fe and Co.

The transition metal may be added, for example, as an oxide, sulfate or tungstate, preference being given to the two latter possibilities. In this case, the transition metal salt may also be the sulfate or tungstate source. It is also possible to use mixtures of the compounds mentioned.

The concentration of the transition metal compound in the catalyst is, if present, preferably from 0.1 to 20% by weight, more preferably from 1 to 10% by weight, based on the metal oxide.

The catalysts are preferably arranged in a fixed bed and therefore preferably in piece form, for example in the form of tablets (5 mm×5 mm, 5 mm×3 mm, 3 mm×3 mm, 1.5×1.5 mm), rings (7 mm×7 mm×3 mm, 5 mm×5 mm×3 mm, 5 mm×2 mm×2 mm) or extrudates (diameter 1.5 mm, diameter 3 mm, diameter 5 mm). The above size data are merely by way of example and do not constitute any restriction.

Specifically, step (a) is performed preferably in accordance with the invention in such a way that a hydrocarbon stream comprising 2-butenes, n-butane and at most small amounts of 1-butene and isobutene is converted over the catalysts mentioned, preferably in the liquid phase.

Such hydrocarbon streams are generally obtained from a steamcracker, an FCC plant (Fluid Catalytic Cracking) or a butane dehydrogenation, and, if appropriate, processed further for use in the process according to the invention.

Suitable C₄ hydrocarbon streams are, for example, mixtures with the following composition:

butane: from 10 to 90% by weight;

butene: from 90 to 10% by weight,

where the butene fraction may have the following composition:

1-butene: from 0 to 5% by weight

cis-2-butene: from 1 to 50% by weight

trans-2-butene: from 1 to 99% by weight

isobutene: from 0 to 5% by weight,

and the butane fraction may have the following composition:

n-butane: from 70 to 100% by weight

isobutane: from 0 to 30% by weight.

A preferred feedstock used is a butane-containing C₄ hydrocarbon mixture obtained from so-called raffinate II or raffinate III, which is obtained from the C₄ cut of steamcrackers or FCC plants after removing highly unsaturated hydrocarbons such as diolefins, especially 1,3-butadiene, or acetylene, and then removing the isobutene present therein. In raffinate III, some of the n-butenes have additionally also been removed.

The details of the provision of suitable streams rich in 2-butenes are disclosed in detail later in the description.

The C₄ hydrocarbon streams may be freed, for example, in the manner known from DE-A 39 14 817, of butadiene, sulfur-containing and oxygen-containing compounds, such as alcohols, aldehydes, ketones or ethers, by selective hydrogenation or adsorption on a molecular sieve.

The dimerization reaction takes place generally at temperatures of from 10 to 280° C., preferably from 10 to 190° C. and in particular from 20 to 130° C., and a pressure of generally from 1 to 300 bar, preferably from 15 to 100 bar and in particular from 5 to 50 bar. The pressure is appropriately selected in such a way that the feed hydrocarbon mixture is present in a liquid or supercritical state at the temperature established. The reactor is generally a cylindrical reactor charged with the catalyst, which is flowed through by the liquid reaction mixture, for example, from the top downward. The dimerization process can be performed in an individual reactor up to the desired end conversion of the butenes, and the catalyst may be arranged in a single fixed bed or a plurality of fixed beds in the reactor. Alternatively, the process can be performed by using a reactor battery composed of a plurality, preferably two, reactors connected in series, in which case the dimerization of the butenes in the reaction mixture in a preferred embodiment is operated only up to a partial conversion as it passes through the reactor or reactors connected upstream of the last reactor of the battery, and the desired end conversion is achieved only as the reaction mixture passes through the last reactor of the battery. In the individual reactors of the reactor battery, the oligomerization catalyst may be arranged in a single fixed catalyst bed or in a plurality of fixed catalyst beds.

Moreover, in the individual reactors of the reactor battery, different reaction conditions with regards to pressure and/or temperature may be established within the abovementioned pressure and temperature ranges. It is also possible to use different dimerization catalysts in the individual reactors of the battery, although the use of the same catalyst in all reactors of the battery is preferred.

In a preferred embodiment, butane removed from the reaction mixture and unconverted butene are recycled into the dimerization reaction (see, for example, WO 99/25668).

When a single dimerization reactor is used in this embodiment, the recycled C₄ hydrocarbon mixture which has been depleted in butenes and comprises predominantly butanes is added to the use hydrocarbon mixture advantageously before it enters the reactor. However, it is also possible to introduce the use hydrocarbon mixture and the recycled C₄ hydrocarbon mixture into the oligomerization reactor via separate lines. When the catalyst in the oligomerization reactor is arranged in a plurality of fixed beds, the recycled hydrocarbon stream may be divided and introduced into the reactor at a plurality of points, for example upstream of the first fixed bed in flow direction of the reaction mixture and/or between the individual fixed catalyst beds. The same applies when a reactor battery is used, in which case the recycled hydrocarbon stream can either be fed completely to the first reactor of the battery or can be distributed via a plurality of feed lines between the individual reactors of the battery, as described for the case of an individual reactor. After leaving the single-stage or multistage reaction zone, the oligomers formed are separated in a manner known per se from the unconverted C₄ hydrocarbons, and these C₄ hydrocarbons are recycled completely or for the most part, preferably in such an amount that the content of oligomers in the converted reaction mixture does not exceed 30% by weight, preferably 20% by weight, at any point in the reactor, or, in the case of use of a reactor battery, at any point in the reactor battery. Expressed in other words, the preferred recycling of the C₄ hydrocarbon mixtures should preferably be controlled in such a way that the oligomer content of the converted reaction mixture does not exceed 30% by weight, preferably 20% by weight, at any point in the reactor, or, in the case of use of a reactor battery, at any point in the reactor battery, and the oligomer content in the converted reaction mixture advantageously does not go below 10% by weight when it leaves the reactor or, in the case of use of a reactor battery, when it leaves the reactor battery. To achieve such an oligomer content of the converted reaction mixture, a weight ratio of recycle stream to freshly supplied use hydrocarbon stream of generally from 0 to 10, preferably from 0 to 7, in particular from 0 to 4 is established, these data relating to the steady state of the reaction system.

There is in principle no lower limit for the dimer content in the reaction mixture, but the process becomes uneconomic in the case of selection of a very low dimer content owing to the recycle stream becoming excessively large. Therefore, the dimer content generally does not go below a lower limit of 10% by weight of dimers in the converted reaction mixture before its workup.

In one embodiment, the process is performed in adiabatic operation.

As opposed to isothermal operation in which the amount of heat formed in an exothermic reaction is removed by cooling by means of cooling or thermostatting apparatus, such as thermostatting baths, cooling jackets or heat exchangers, and the temperature in the reactor is thus kept constant, i.e. isothermal, adiabatic operation is understood to mean an operating mode in which the amount of heat released in an exothermic reaction is absorbed by the reaction mixture in the reactor and no cooling by cooling apparatus is employed. It is obvious that purely adiabatic operation in the theoretical and academic sense of the term “adiabatic” could be accomplished in industry only with an uneconomically high level of complexity, since a portion, even though negligibly small, of the amount of heat released in the exothermic reaction is virtually unavoidably also absorbed by the reactor body and released to the environment by heat conduction and heat emission. In the technical sense, adiabatic operation or operating mode is therefore understood to mean operation or an operating mode in which, apart from the portion of the heat of reaction released from the reactor to the environment by natural heat conduction and heat emission, all of the heat of reaction is absorbed by the reaction mixture and removed from the reactor together with it. In the industrial sense, the term “quasi-adiabatic” operation is therefore also used synonymously to adiabatic operation.

Since the exothermic reaction in the case of the present dimerization step occurs solely as a result of the contact of the butenes with the dimerization catalyst, and heat is thus released only in the region of the catalyst bed, the reaction temperature in the catalyst bed and hence also the temperature in the reactor can in principle be controlled by the supply of the reactants. The more butene is converted over the catalyst, the more the temperature in the catalyst bed increases, i.e. the higher the reaction temperature becomes. Since no heat is removed via cooling apparatus in such an adiabatic operating mode, the heat of reaction formed in the dimerization is removed virtually solely by the reaction mixture flowing through the catalyst bed. Such an operating mode would, without additional measures, when the starting hydrocarbon mixtures to be used in accordance with the invention are used, greatly limit the throughput and hence the conversion per unit time, based on one volume unit of the oligomerization catalyst used, since an increase in the conversion would result in a large rise in the reaction temperature.

The recycled hydrocarbon stream may have been cooled to a lower temperature before it is added to the freshly supplied use hydrocarbon stream, or, in the case of direct introduction into the oligomerization catalyst, before its introduction, as a result of which the removal of the heat of reaction can additionally be improved. The quasi-adiabatic operating mode also comprises a process configuration in which the conversion of the butenes to dimers is distributed within a reactor battery composed of two or more, preferably two, dimerization reactors, and the partly converted reaction mixture, after leaving one reactor and before entering the next reactor of the battery, is cooled by means of conventional cooling apparatus such as cooling jackets or heat exchangers.

In a further preferred embodiment of stage (a), the crude product stream, after leaving the single-stage or multistage reaction zone, is divided into a first and a second product substream.

The first product substream is worked up in a manner known per se, preferably by distillation, to the dimers formed.

At the same time, residual amounts of the unconverted alkenes and any accompanying alkanes are removed as a “purge stream” and, as described below, used as starting materials of a butene dehydrogenation.

The purge stream may also be recycled partly or fully into the first reactor. With its low content of reactive alkene, its action consists, for example, in enlarging the stream through the reactor, i.e. in diluting the alkene, and hence ultimately contributing to the temperature control in the reactor. Moreover, it is thus possible to control the upper limit for the dimer content in the product stream more easily.

The second product substream is recycled into the process with virtually unchanged composition. The temperature of the second product substream can be adjusted to the desired temperature before it is supplied into the reactor with apparatus known for this purpose, such as heat exchangers.

Since processes are typically optimized to feedstocks of certain properties and composition, it is generally also required for a sufficient conversion to increase the content of the alkene in the second product substream back to the starting value at the reactor inlet, or close to it. To this end, preference is given to metering fresh alkene which can be mixed with the alkanes into the second substream.

The person skilled in the art can determine what ratio the stream of the fresh alkene and any recycled proportion of the purge stream have to be in relative to the second substream before feeding into the reactor easily by means of simple preliminary experiments with regard to the desired dimer yield and selectivity and the temperature to be established for this purpose in the reactor interior.

The second product substream and the fresh stream of the alkene can be fed into the reactor in such a way that the streams are passed into the reactor simultaneously and individually, for instance via separate lines, or after preceding mixing.

Before they are fed into the reactor, the temperature of each individual stream or the mixture of the streams of the feedstocks can be adjusted with apparatus known per se for this purpose, such as heat exchangers.

When the catalyst is arranged in a plurality of fixed beds in the reactor, the mixed feedstock streams can be divided and introduced into the reactor at a plurality of points, for example upstream of a first fixed bed in flow direction of the reaction mixture and/or between individual fixed catalyst beds. When a reactor battery, for example, is used, it is possible to feed to the mixed feedstock streams either completely to the first reactor of the battery or to divide them via several feed lines between the individual reactors of the battery, as described for the case of the individual reactor.

The process described affords contents of dimers of from 5 to 100% by weight, preferably from 10 to 60% by weight and in particular from 15 to 30% by weight, based on the overall product stream. To achieve such a dimer content, a weight ratio of recycle stream to freshly supplied use hydrocarbon stream of generally from 0.5 to 10, preferably from 1 to 7, in particular from 1 to 4, is established, these data relating to the steady state of the reaction system.

In contrast to adiabatic operation, in the likewise preferred isothermal operation, for technical purposes, the discharge of the heat of reaction from the reactor by means of cooling or thermostatting apparatus is enhanced in a controlled manner over and above the degree caused by natural heat conduction and heat emission, the heat of reaction generally being absorbed initially by a heat carrier medium, the coolant, before it is released to the environment or, for example, in the case of use of heat exchangers, utilized for the heating of substances or for energy generation.

In a further preferred embodiment of stage (a), which is described, for example, in DE-A 100 55 036, the 2-butenes are dimerized in a process which permits reaction with essentially constant temperature without any requirement for cooling or thermostatting apparatus in the reactor, and in which
a) a feed which is present in essentially monophasic liquid form and comprises the 2-butenes is provided,
b) the feed is passed under essentially adiabatic conditions over a bed of a particulate heterogeneous, preferably nickel-containing, catalyst,
c) the feed, preferably counter to the direction of gravity, being passed over the bed of the catalyst and the pressure being selected such that the feed, owing to the heat of reaction, evaporates partly to form a vapor phase, and the vapor phase is discharged from the bed of the catalyst in cocurrent with the liquid phase.

In addition to the 2-butenes, the feed preferably comprises at least one inert solvent. The 2-butene content in the feed is preferably from about 10 to 90% by weight, and the content of diluent is preferably from about 90 to 10% by weight, based in each case on the total mass of the feed.

Suitable diluents are, for example, the constituents of the use stream other than 2-butenes saturated hydrocarbons. In general, the diluent has a boiling point which differs by a maximum of 15° C. from that of the olefin to be oligomerized. Especially suitable are alkanes having from 2 to 6 carbon atoms, for example ethane, propane, n-butane, n-pentane or n-hexane.

In this preferred variant, the starting material is a feed present in monophasic liquid form. Although the dimerization of olefins proceeds exothermically, the feed has to have such a temperature that the reaction commences spontaneously on contact with the catalyst. In general, it is therefore necessary to preheat the feed which has been brought to reaction pressure. The temperature of the feed is selected such that the boiling point is not quite attained at this pressure. On contact with the catalyst, the reaction liquid begins to boil as a result of the heat released in the dimerization. The energy needed for the change in state of matter is withdrawn by the reaction liquid. The reaction temperature corresponds to the boiling temperature of the reaction liquid. The boiling point is pressure-dependent, since the pressure of the steam corresponds to the external pressure at the boiling point. A pressure increase leads to a rise in the boiling temperature; a decrease in the pressure leads to a fall in the boiling temperature. The reaction temperature can therefore be adjusted by controlling the pressure.

The process is conducted in such a way that at least a small portion of the reaction liquid evaporates during the reaction, but complete evaporation of the reaction liquid is prevented. In addition to the control of the pressure, this can be achieved in particular by controlling the cross-sectional loading of the catalyst and/or a variation in the composition of the 2-butene-containing feed. For instance, the concentration in the feed can be decreased by additionally using inert diluents, and the maximum expected exothermicity can thus be restricted.

When the diluent is the lowest-boiling component, it is evaporated preferentially during the reaction. Additional use of a sufficiently large amount of diluent allows complete evaporation of the reaction liquid to be prevented. The exothermicity and accordingly the portion of the reaction liquid evaporated during the reaction can also be influenced by varying the cross-sectional loading of the catalyst bed, an increase in the cross-sectional loading generally leading to greater exothermicity.

The process is preferably conducted in such a way that, especially by suitable selection of the cross-sectional loading and/or of the composition of the feed, the concentration of dimers in the liquid phase leaving the bed of the catalyst is within the range from about 5 to 80% by weight, preferably from about 10 to 50% by weight and in particular from about 15 to 30% by weight, based on the liquid phase.

The mixture obtained from stage (a) generally also comprises by-products such as trimers and tetramers, which are removed if appropriate in a purification step. Preference is given to distillative purification.

### DEHYDROGENATING AROMATIZATION/STAGE (B)

In stage (b), the mixture which is obtained in (a) and comprises predominantly 3,4- and/or 2,3-dimethylhexenes is converted to o-xylene in a dehydrogenating aromatization. The reaction is performed heterogeneously with a catalyst.

Suitable catalysts are, for example:
a) a highly dehydrogenating metal, preferably from the platinum group, especially platinum, in combination with a nonacidic support, preferably a crystalline, microporous material, especially zeolites, SAPOs or ALPOs, which preferably comprise In, Sn, Tl or Pb.
Such catalysts are described, for example, in U.S. Pat. No. 4,910,357.
b) a catalyst which comprises a noble metal selected from the elements of transition group VIII of the Periodic Table of the Elements, especially palladium, platinum or rhodium and/or rhenium and/or tin, on one or more ceramic oxides of elements from the third and fourth main group and the fourth transition group (group IVB) of the elements and of the lanthanides, especially Al₂O₃, SiO₂, ZrO₂, TiO₂, La₂O₃ and Ce₂O₃.
Such catalysts are described, for example, in WO 97/40931.
c) Mo₂C catalysts, preferably on an SiO₂ support, particular preference being given to Mo₂C concentrations on the support of from 1 to 20% by weight, in particular from 2 to 10% by weight.
Such catalysts are described, for example, in Catalysis Letters 101 (2005) p. 235-239.

Preference is given to catalysts of group (b) which are described in detail below:

In addition to the elements mentioned above for the catalysts of group (b), it is possible to use further elements; in particular, rhenium and/or tin should be understood as additions to the elements of transition group VIII. Another constituent is the addition of or the doping with either compounds of the third main or transition group (IIIA or IIIB) or basic compounds such as alkali earths, alkaline earths or rare earths, or their compounds which can be converted to the corresponding oxides at temperatures above 400° C. Simultaneous doping with a plurality of the elements mentioned or their compounds is possible. Suitable examples are potassium and lanthanum compounds. In addition, the catalyst may be admixed with sulfur, tellurium, arsenic, antimony or selenium compounds, which in many cases bring about an increase in the selectivity, presumably by partial “poisoning” (moderators).

To prepare the catalysts (b), it is possible to use so-called amphoteric ceramic oxides, i.e. in particular oxides of zirconium and of titanium or mixtures thereof; also suitable are corresponding compounds which can be converted to these oxides by calcining. They can be prepared by known processes, for example by the sol-gel process, precipitation of the salts, dewatering of the corresponding acids, dry mixing, slurrying or spray-drying.

Suitable supports are all modifications of zirconium oxide and titanium oxide. However, it has been found to be advantageous for the preparation of catalysts on the basis of ZrO₂ when the proportion of monoclinic ZrO₂ detectable by X-ray diffraction is more than 90%. Monoclinic ZrO₂ is characterized in the X-ray diffractogram by two strong signals in the two-theta range of about 28.2 and 31.5.

The doping with a basic compound can be effected during the preparation, for example by coprecipitation, or subsequently, for example by impregnating the ceramic oxide with an alkali metal or alkaline earth metal compound or a compound of an element of the third transition group or a rare earth metal compound.

The content of alkali metal or alkaline earth metal, metal of main or transition group III, rare earth metal or zinc is generally up to 20% by weight, preferably between 0.1 and 15% by weight, more preferably between 0.5 and 10% by weight. The alkali metal and alkaline earth metal providers used are generally compounds which can be converted to the corresponding oxides by calcining. Suitable examples are hydroxides, carbonates, oxalates, acetates, nitrates or mixed hydroxycarbonates, or alkali metals and alkaline earth metals.

When the ceramic support is doped additionally or exclusively with a metal of the third main or transition group, the starting materials in this case too should be compounds which can be converted to the corresponding oxides by calcining. When lanthanum is used, for example, lanthanum compounds which comprise organic anions, such as lanthanum acetate, lanthanum formate or lanthanum oxalate, are suitable.

The noble metal constituents may be applied in different ways. For example, the support can be impregnated or sprayed with a solution of a corresponding compound of the noble metal, or of rhenium or tin. Suitable metal salts for preparing such solutions are, for example, the nitrates, halides, formates, oxalates, acetates of the noble metal compounds. It is also possible to use complex anions, or acids of these complex anions, such as H₂PtCl₆. Particularly suitable compounds for preparing the inventive catalysts have been found to be PdCl₂, Pd(OAc)₂, Pd(NO₃)₂ and Pt(NO₃)₂.

The use of noble metal sols with one or more components in which the active component is already present completely or partly in the reduced state also leads to the catalysts (b).

When noble metal sols are used, these are prepared beforehand in a customary manner, for example by reduction of a metal salt or of a mixture of a plurality of metal salts in the presence of a stabilizer such as polyvinylpyrrolidone, and then applied to it either by impregnating or spraying the support. The preparation technique is disclosed in the German patent application 1 95 00 366.7.

The content in the catalyst of elements of transition group VIII and/or rhenium or tin may, for example, be from 0.005 to 5% by weight, preferably from 0.01 to 2% by weight, more preferably from 0.1 to 1.5% by weight. When rhenium or tin is used in addition, its ratio to the noble metal constituent may, for example, be from 0.1:1 to 20:1, preferably from 1:1 to 10:1.

The moderating additives used (according to the common conception of partial poisoning of the catalyst) may, if required, be compounds of sulfur, of tellurium, of arsenic or of selenium. The addition of carbon monoxide during the operation of the catalyst is also possible. The use of sulfur has been found to be particularly advantageous, which is conveniently applied in the form of ammonium sulfide, (NH₄)₂S. The molar ratio of noble metal components to moderating compound may be from 1:0 to 1:10, preferably from 1:1 to 1:0.05.

The catalyst may be used in a fixed bed in the reactor or, for example, in the form of a fluidized bed and have a corresponding shape. Suitable shapes are, for example, spall, tablets, monoliths, spheres or extrudates (strands, wagonwheels, stars, rings).

The catalyst preparations generally have a BET surface area of up to 500 m²/g, usually from 10 to 300 m²/g, more preferably from 20 to 300 m²/g. The pore volume is generally between 0.1 and 1 ml/g, preferably from 0.15 to 0.6 ml/g, more preferably from 0.2 to 0.4 ml/g. The mean pore diameter of the mesopores determinable by Hg penetration analysis is generally between 8 and 60 nm, preferably between 10 and 40 nm. The proportion of the pores having a width of more than 20 nm varies generally between 0 and 60%; it has been found to be advantageous to use supports having a macropore fraction (i.e. pores of width more than 20 nm) of more than 10%.

A preferred group of catalysts of group (b) is that of the systems described in EP-A 1 074 301 which will be described in detail below.

These are catalysts with bimodal pore radius distribution which comprise
a) from 10 to 99.9% by weight of zirconium dioxide and
b) from 0 to 60% by weight of aluminum oxide, silicon oxide and/or titanium oxide and
c) from 0.1 to 30% by weight of at least one element of main group I or II, of an element of transition group III, of an element of transition group VIII of the Periodic Table of the Elements, cerium, lanthanum and/or tin,
   with the proviso that the sum of the percentages by weight is 100.

These catalysts comprise
a) from 10 to 99.9% by weight, preferably from 20 to 98% by weight, particularly preferably from 30 to 95% by weight, of zirconium dioxide of which from 50 to 100% by weight, preferably from 60 to 99% by weight, particularly preferably from 70 to 98% by weight, is in the monoclinic and/or tetragonal modification and
b) from 0.1 to 30% by weight, preferably from 0.5 to 25% by weight, particularly preferably from 1 to 20% by weight of silicon dioxide and
c) from 0 to 60% by weight, preferably from 0.1 to 50% by weight, particularly preferably from 1 to 40% by weight, in particular from 5 to 30% by weight, of aluminum oxide silicon dioxide and/or titanium dioxide in the form of rutile or anatase, and
d) from 0.1 to 10% by weight, preferably from 0.2 to 8% by weight, particularly preferably from 0.5 to 5% by weight, of at least one element selected from main group I or II, transition groups III and VIII and from the transition group of the Periodic Table of the Elements, cerium, lanthanum and/or tin,
   where the sum of the percentages by weight is 100.

The catalysts preferably consist of the composition specified.

The catalysts comprise from 70 to 100%, preferably from 75 to 98%, particularly preferably from 80 to 95%, of larger pores than 20 nm, preferably between 40 and 5000 nm.

To produce the catalysts, use can be made of precursors of the oxides of zirconium, titanium, lanthanum, cerium, silicon and aluminum (forming the support) which can be converted by calcination into the oxides. These can be prepared by known methods, for example by the sol-gel process, precipitation of the salts, dehydration of the corresponding acids, dry mixing, slurrying or spray drying. For example, a ZrO₂.xAl₂O₃.xSiO₂ mixed oxide can be prepared by first preparing a water-rich zirconium oxide of the general formula ZrO₂.xH₂O by precipitation of a suitable zirconium-containing precursor. Suitable zirconium precursors are, for example, Zr(NO₃)₄, ZrOCl₂ or ZrCl₄. The precipitation itself is carried out by addition of a base such as NaOH, Na₂CO₃ and NH₃ and is described, for example, in EP-A 849 224.

To prepare a ZrO₂.xSiO₂ mixed oxide, the Zr precursor obtained as above can be mixed with an Si-containing precursor. Well suited SiO₂ precursors are, for example, water-containing sols of SiO₂ such as Ludox®. The two components can be mixed, for example, by simple mechanical mixing or by spray drying in a spray tower.

When using mixed oxides, it is possible to influence the pore structure in a targeted way. The particle sizes of the various precursors influence the pore structure. Thus, for example, macropores can be generated in the microstructure by use of Al₂O₃ having a low loss on ignition and a defined particle size distribution. An aluminum oxide which has been found to be useful for this purpose is Puralox (Al₂O₃ having a loss on ignition of about 3%).

To prepare a ZrO₂.xSiO₂.xAl₂O₃ mixed oxide, the SiO₂.xZrO₂ powder mixture obtained as described above can be admixed with an Al-containing precursor. This can be carried out, for example, by simple mechanical mixing in a kneader. However, a ZrO₂.xSiO₂.xAl₂O₃ mixed oxide can also be prepared in a single step by dry mixing of the individual precursors.

Compared to pure ZrO₂, the mixed oxides have the advantage, inter alia, that they can be shaped easily. For this purpose, the powder mixture obtained is admixed in a kneader with a concentrated acid and can then be converted into a shaped body, e.g. by means of a ram extruder or a screw extruder.

A further possible way of producing the support having a specific pore radius distribution for the catalysts mentioned, in a targeted manner, is to add, during the preparation, various polymers which can be partly or completely removed by calcination so as to form pores in defined pore radius ranges. The mixing of the polymers and the oxide precursors can, for example, be carried out by simple mechanical mixing or by spray drying in a spray tower.

The use of PVP (polyvinylpyrrolidone) has been found to be particularly advantageous for producing the supports having a bimodal pore radius distribution. If PVP is added during a production step to one or more oxide precursors of the elements Zr, Ti, La, Ce, Al or Si, macropores in the range from 200 to 5000 nm are formed after calcination. A further advantage of the use of PVP is that the support can be shaped more readily. Thus, extrudates having good mechanical properties can be produced from freshly precipitated water-containing ZrO₂.xH₂O which has previously been dried at 120° C. when PVP and formic acid are added, even without further oxide precursors.

The mixed oxide supports of the catalysts generally have higher BET surface areas after calcination than do pure ZrO₂ supports. The BET surface areas of the mixed oxide supports are generally from 40 to 300 m²/g, preferably from 50 to 200 m²/g, particularly preferably from 60 to 150 m²/g. The pore volume of the catalysts of the present invention used is usually from 0.1 to 0.8 ml/g, preferably from 0.2 to 0.6 ml/g. The mean pore diameter of the catalysts of the present invention, which can be determined by Hg porosimetry, is from 5 to 20 nm, preferably from 8 to 18 nm. Furthermore, it is advantageous for from 10 to 80% of the pore volume to be made up by pores >40 nm.

The calcination of the mixed oxide supports is advantageously carried out after the application of the active components and is carried out at from 400 to 700° C., preferably from 500 to 650° C., particularly preferably from 560 to 620° C. The calcination temperature should usually be at least as high as the reaction temperature of the dehydrogenation.

The catalysts have a bimodal pore radius distribution. The pores are mostly in the range up to 20 nm and in the range from 40 to 5000 nm. Based on the pore volume, these pores make up at least 70% of the pores. The proportion of pores less than 20 nm is generally from 20 to 60%, while the proportion of pores in the range from 40 to 5000 nm is generally likewise from 20 to 60%.

The doping of the mixed oxides with a basic compound can be carried out either during their preparation, for example by coprecipitation, or subsequently, for example by impregnation of the mixed oxide with an alkali metal compound or alkaline earth metal compound or a compound of transition group III or a rare earth metal compound. Particularly suitable dopants are K, Cs and La.

The application of the dehydrogenation-active component, which is usually a metal of transition group VIII, is generally carried out by impregnation with a suitable metal salt precursor which can be converted into the corresponding metal oxide by calcination. As an alternative to impregnation, the dehydrogenation-active component can also be applied by other methods, for example spraying the metal salt precursor onto the support. Suitable metal salt precursors are, for example, the nitrates, acetates and chlorides of the appropriate metals, or complex anions of the metals used. Preference is given to using platinum as H₂PtCl₆ or Pt(NO₃)₂. Solvents which can be used for the metal salt precursors are water and organic solvents. Particularly suitable solvents are lower alcohols such as methanol and ethanol.

Further suitable precursors when using noble metals as dehydrogenation-active component are the corresponding noble metal sols which can be prepared by one of the known methods, for example by reduction of a metal salt with a reducing agent in the presence of a stabilizer such as PVP. The preparation technique is dealt with comprehensively, for example in the German Patent Application DE-A 195 00 366.

As alkali metal and alkaline earth metal precursors, use is generally made of compounds which can be converted into the corresponding oxides by calcination. Examples of suitable precursors are hydroxides, carbonates, oxalates, acetates or mixed hydroxycarbonates of the alkali metals and alkaline earth metals.

If the mixed oxide support is additionally or exclusively doped with a metal of main group III or transition group III, the starting materials in this case too should be compounds which can be converted into the corresponding oxides by calcination. If lanthanum is used, suitable starting compounds are, for example, lanthanum oxide carbonate, La(OH)₃, La₂(CO₃)₃, La(NO₃)₃ or lanthanum compounds Containing organic anions, e.g. lanthanum acetate, lanthanum formate or lanthanum oxalate.

The dehydrogenating aromatization (b) is preferably performed in the gas phase.

The aromatization is performed generally at temperatures of from 300 to 800° C., preferably from 400 to 600° C., more preferably from 450 to 550° C., and at pressures of from 100 mbar to 100 bar, preferably from 1 to 30 bar, more preferably from 1 to 10 bar, with an LHSV (Liquid Hourly Space Velocity) of from 0.01 to 100 h⁻¹, preferably from 0.1 to 20 h⁻¹. In addition to the hydrocarbon mixture, diluents such as CO₂, N₂, noble gases or steam may be present. It is likewise possible to add hydrogen if required, in which case the volume ratio of hydrogen to hydrocarbon (gases) may be from 0.1 to 100, preferably from 0.1 to 20. The hydrogen added, or that which has been formed in the dehydrogenation and, if appropriate, recycled, may be used to remove carbon which accumulates on the surface of the catalyst with increasing reaction time.

In addition to the continual (continuous) addition of a gas which prevents coke deposition during the reaction, there is the possibility of regenerating the catalyst from time to time by passing hydrogen or air over it. The regeneration itself takes place at temperatures in the range from 300 to 900° C., preferably from 400 to 800° C., with a free oxidizing agent, preferably with air or air-nitrogen mixtures, and/or in reducing atmosphere, preferably with hydrogen. The regeneration can be operated at atmospheric, reduced or superatmospheric pressure. Suitable pressures are, for example, from 500 mbar to 100 bar.

The workup can be effected by distillation; unconverted reaction mixture is preferably recycled into the reaction circuit.

Obtaining a Starting Stream Rich in 2-butenes

The stream which is rich in 2-butenes and is used for the dimerization (a) comprises preferably at least 20% by weight, more preferably at least 30% by weight, in particular from 40 to 100% by weight, of 2-butenes. It comprises generally from 0 to 10% by weight, preferably from 0 to 2% by weight, more preferably from 0 to 1% by weight, of 1-butene. To obtain such a stream, the starting material in a preferred variant of the process according to the invention is a butane-rich stream which, in a first stage (a1), is dehydrogenated under catalytic conditions to give a butanes/butenes/butadiene stream which is then converted in a second stage (a2) by selectively hydrogenating under isomerizing conditions to give a stream rich in 2-butenes.

This is followed by the dimerization to the desired dimethylhexenes as stage (a3).

Stage (a1) comprises the partial stages of:
(a1i) providing a use gas stream comprising n-butane;
(a1ii) feeding the n-butane-comprising use gas stream into at least one dehydrogenation zone and catalytically hydrogenating n-butane to obtain a product gas stream comprising n-butane, 1-butene, 2-butene, butadiene, low-boiling secondary constituents and hydrogen, with or without steam and nitrogen;
(a1iii) removing hydrogen, the low-boiling secondary constituents and if appropriate water to obtain a C₄ product gas stream essentially consisting of n-butane, 1-butene, 2-butene and butadiene.

In the first process part (a1i), an n-butane-comprising use gas stream is provided. Typically, the raw material used is n-butane-rich gas mixtures such as liquefied petroleum gas (LPG). LPG comprises essentially C₂-C₅ hydrocarbons. In addition, it also comprises traces of methane and C₆⁺ hydrocarbons. The composition of LPG can vary greatly. Advantageously, the LPG used comprises at least 80% by weight of butanes.

Alternatively, it is also possible to use a C₄ hydrocarbon stream from crackers or refineries as the use gas stream.

In one variant of the process, the provision of the n-butane-comprising dehydrogenation use gas stream comprises the steps of
providing a liquefied petroleum gas (LPG) stream,
removing propane and, if appropriate, methane, ethane and C₅⁺ hydrocarbons (mainly pentanes, additionally also hexanes, heptanes, benzene and toluene) from the LPG stream to obtain a stream comprising butanes (n-butane and isobutane),
removing isobutane from the stream comprising butanes to obtain the n-butane-comprising use gas stream, and if appropriate isomerizing the isobutane removed to give an n-butane/isobutane mixture and recycling the n-butane/isobutane mixture into the isobutane removal.

Propane and, if appropriate, methane, ethane and C₅⁺ hydrocarbons are removed, for example, in one or more customary rectifying columns. For example, low boilers (methane, ethane, propane) are removed via the top of one column and, in the same column or a second column, high boilers (C₅⁺ hydrocarbons) are removed at the bottom of the column. A stream comprising butanes (n-butane and isobutane) is obtained, from which isobutane is removed, for example in a customary rectifying column. The remaining n-butane-comprising stream is used for the subsequent butane dehydrogenation.

The isobutane stream removed is preferably subjected to an isomerization. To this end, the isobutane-comprising stream is fed into an isomerization reactor. The isomerization of isobutane to n-butane can be performed as described in GB-A 2 018 815. An n-butane/isobutane mixture is obtained, which is fed into the n-butane/isobutane separating column.

In process stage (a1ii), the n-butane-comprising use gas stream is fed into a dehydrogenation zone and subjected to a catalytic dehydrogenation. In this stage, n-butane is dehydrogenated in a dehydrogenation reactor over a dehydrogenation-active catalyst partially to give 1-butene and 2-butene, and butadiene is also formed. In addition, hydrogen and small amounts of methane, ethane, ethene, propane and propene are obtained. Depending on the method of dehydrogenation, for example with autothermal addition of O₂-containing gas, carbon oxides (CO, CO₂), water and nitrogen may also be present in the product gas mixture of the catalytic n-butane dehydrogenation. In addition, unconverted n-butane is present in the product gas mixture.

The catalytic n-butane dehydrogenation can be performed with or without oxygenous gas as a cofeed.

The catalytic n-butane dehydrogenation may in principle be carried out in all reactor types and methods known from the prior art. A comprehensive description of dehydrogenation processes suitable in accordance with the invention is also present in “Catalytic® Studies Division, Oxidative Dehydrogenation and Alternative Dehydrogenation Processes” (Study Number 4192 OD, 1993, 420 Ferguson Drive, Mountain View, Calif. 94043-5272, USA).

A suitable reactor form is the fixed bed tubular reactor or tube bundle reactor. In these reactors, the catalyst (dehydrogenation catalyst and, when working with oxygen as a cofeed, if appropriate a specific oxidation catalyst) is disposed as a fixed bed in a reaction tube or in a bundle of reaction tubes. The reaction tubes are typically heated indirectly by combustion of a gas, for example a hydrocarbon such as methane, in the space surrounding the reaction tubes. It is favorable to apply this indirect form of heating only to the first approx. 20 to 30% of the length of the fixed bed and to heat the remaining bed length to the required reaction temperature by virtue of the radiative heat released in the course of the indirect heating. Typical reaction tube internal diameters are from about 10 to 15 cm. A typical dehydrogenation tube bundle reactor comprises from approx. 300 to 1000 reaction tubes. The temperature in the reaction tube interior varies typically within the range from 300 to 1200° C., preferably within the range from 400 to 1000° C. The working pressure is typically between 0.5 and 8 bar, frequently between 1 and 2 bar when a low steam dilution is used (corresponding to the Linde process for propane dehydrogenation), or else between 3 and 8 bar when a high steam dilution is used (corresponding to the so-called “steam active reforming process” (STAR process) for dehydrogenating propane or butane of Phillips Petroleum Col., see U.S. Pat. No. 4,902,849, U.S. Pat. No. 4,996,387 and U.S. Pat. No. 5,389,342). Typical gas hourly space velocities (GHSV) are from 500 to 2000 h⁻¹, based on hydrocarbon used. The catalyst geometry may, for example, be spherical or cylindrical (hollow or solid).

The catalytic n-butane dehydrogenation may also, as described in Chem. Eng. Sci. 1992 b, 47 (9-11) 2313, be carried out under heterogeneous catalysis in a fluidized bed. In this case, two fluidized beds are appropriately operated alongside one another, of which one is generally in the state of regeneration. The working pressure is typically from 1 to 2 bar, the dehydrogenation temperature generally from 550 to 600° C. The heat required for the dehydrogenation is introduced into the reaction system by the dehydrogenation catalyst being preheated to the reaction temperature. The admixing of an oxygen-comprising cofeed allows the preheater to be dispensed with, and the heat required to be generated directly in the reactor system by combustion of hydrogen and/or hydrocarbons in the presence of oxygen. If appropriate, a hydrogen-comprising cofeed may additionally be admixed.

The catalytic n-butane dehydrogenation may be carried out with or without oxygenous gas as a cofeed in a tray reactor. This comprises one or more successive catalyst beds. The number of catalyst beds may be from 1 to 20, appropriately from 1 to 6, preferably from 1 to 4 and in particular from 1 to 3. The catalyst beds are preferably flowed through radially or axially by the reaction gas. In general, such a tray reactor is operated with a fixed catalyst bed. In the simplest case, the fixed catalyst beds are arranged axially in a shaft furnace reactor or in the annular gaps of concentrically arranged cylindrical grids. A shaft furnace reactor corresponds to one tray. The performance of the dehydrogenation in a single shaft furnace reactor corresponds to a preferred embodiment, in which it is possible to work with oxygenous cofeed. In a further preferred embodiment, the dehydrogenation is carried out in a tray reactor with 3 catalyst beds. In a method without oxygenous gas as a cofeed, the reaction mixture in the tray reactor is subjected to intermediate heating on its way from one catalyst bed to the next catalyst bed, for example by passing it over heat exchanger surfaces heated with hot gases or by passing it through tubes heated with hot combustion gases.

In a preferred embodiment of the process, the catalytic n-butane dehydrogenation is carried out autothermally. To this end, oxygen is additionally admixed to the reaction gas mixture of the n-butane dehydrogenation in at least one reaction zone and the hydrogen and/or hydrocarbon present in the reaction mixture is at least partly combusted, which generates at least some of the heat of dehydrogenation required in the at least one reaction zone directly in the reaction gas mixture.

Details of this process are described, for example, in WO 2005/042450.

The dehydrogenation catalysts used generally have a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide; particular preference is given to mixtures of zirconium dioxide and silicon dioxide.

The active composition of the dehydrogenation catalysts generally comprises one or more elements of transition group VIII, preferably platinum and/or palladium, more preferably platinum. Furthermore, the dehydrogenation catalysts may comprise one or more elements of main group I and/or II, preferably potassium and/or cesium. The dehydrogenation catalysts may further comprise one or more elements of transition group III including the lanthanides and actinides, preferably lanthanum and/or cerium. Finally, the dehydrogenation catalysts may comprise one or more elements of main group III and/or IV, preferably one or more elements from the group consisting of boron, gallium, silicon, germanium, tin and lead, more preferably tin.

In a preferred embodiment, the dehydrogenation catalyst comprises at least one element of transition group VIII, at least one element of main group I and/or II, at least one element of main group III and/or IV and at least one element of transition group III including the lanthanides and actinides.

For example, all dehydrogenation catalysts which are disclosure by WO 99/46039, U.S. Pat. No. 4,788,371, EP-A 705 136, WO 99/29420, U.S. Pat. No. 5,220,091, U.S. Pat. No. 5,430,220, U.S. Pat. No. 5,877,369, EP 0 117 146, DE-A 199 37 106, DE-A 199 37 105 and DE-A 199 37 107 may be used in accordance with the invention. Particularly preferred catalysts for the above-described variants of autothermal n-butane dehydrogenation are the catalysts according to examples 1, 2, 3 and 4 of DE-A 199 37 107.

Preference is give to carrying out the n-butane dehydrogenation in the presence of steam. The added steam serves as a heat carrier and supports the gasification of organic deposits on the catalysts.

The dehydrogenation catalyst my be regenerated in a manner known per se. For instance, steam may be added to the reaction gas mixture or a gas comprising oxygen may be passed from time to time over the catalyst bed at elevated temperature and the deposited carbon burnt off. The dilution with steam shifts the equilibrium toward the products of dehydrogenation. After the regeneration, the catalyst is reduced with a hydrogenous gas if appropriate.

The n-butane dehydrogenation affords a gas mixture which, in addition to butadiene, 1-butene, 2-butene and unconverted n-butane, comprises secondary constituents. Customary secondary constituents are hydrogen, steam, nitrogen, CO and CO₂, methane, ethane, ethene, propane and propene. The composition of the gas mixture leaving the first dehydrogenation zone can vary greatly depending on the method of dehydrogenation. For instance, when the preferred autothermal dehydrogenation with feeding of oxygen, the product gas mixture of the catalytic dehydrogenation has a comparatively high content of hydrogen.

The product gas stream of the catalytic autothermal n-butane dehydrogenation typically comprises form 0.1 to 15% by volume of butadiene, from 1 to 15% by volume of 1-butene, from 1 to 25% by volume of 2-butene, form 20 to 70% by volume of n-butane, from 1 to 70% by volume of steam, form 0 to 10% by volume of low-boiling hydrocarbons (methane, ethane, ethene, propane and propene), from 0.1 to 40% by volume of hydrogen, from 0 to 70% by volume of nitrogen and from 0 to 5% by volume of carbon oxides.

In one process part (a1iii), the low boiling secondary constituents other than the C₄ hydrocarbons (n-butane, isobutane, 1-butene, cis-/trans-2-butene, isobutene, butadiene) are removed at least partly, but preferably essentially completely, from the product gas stream of the n-butane dehydrogenation to obtain a C₄ product gas stream.

The product gas stream which leaves the dehydrogenation zone is preferably separated into two substreams, in which case only one of the two substreams is subjected to the further process parts and the second substream can be recycled into the dehydrogenation zone. A corresponding procedure is described in DE-A 102 11 275. However, it is also possible for the entire product gas stream of the n-butane dehydrogenation to be subjected to the further process parts.

In one embodiment of the process, water is initially removed from the product gas stream in process part (a1iii). The removal of water can be effected, for example, by condensation by cooling and/or compression of product gas stream b, and can be performed in one or more cooling and/or compression stages. The water removal is typically performed when the n-butane dehydrogenation is performed autothermally or is performed isothermally with feeding of steam (analogously to the Linde or STAR process for dehydrogenating propane), and the product gas stream consequently has a high water content.

The low-boiling secondary constituents can be removed from the product gas stream by customary separating processes such as distillation, rectification, membrane processes, absorption or adsorption.

To remove the hydrogen present in the product gas stream of the n-butane dehydrogenation, the product gas mixture, if appropriate on completion of cooling, can be passed through a membrane which is generally configured as a tube and is permeable only to molecular hydrogen, for example in an indirect heat exchanger. The molecular hydrogen thus removed can, if required, be used at least partly in the dehydrogenation or for subsequent dehydroisomerization, or else be sent to another use.

The carbon dioxide present in the product gas stream of the dehydrogenation can be removed by CO₂ gas scrubbing. The carbon dioxide gas scrubbing may be preceded by a separate combustion stage in which carbon monoxide is oxidized selectively to carbon dioxide.

In a preferred embodiment of the process, the uncondensable or low-boiling gas constituents such as hydrogen, carbon oxides, the low-boiling hydrocarbons (methane, ethane, ethene, propane, propene) and any nitrogen are removed by means of a high-boiling absorbent in an absorption/desorption cycle to obtain a C₄ product gas stream which consists substantially of the C₄ hydrocarbons. In general, the C₄ product gas stream consists to an extent of at least 80% by volume, preferably to an extent of at least 90% by volume, more preferably to an extent of at least 95% by volume, of the C₄ hydrocarbons.

To this end, in an absorption stage, product gas stream—if appropriate after preceding water removal—is contacted with an inert absorbent and the C₄ hydrocarbons are absorbed in the inert absorbent to obtain absorbent laden with C₄ hydrocarbons and an offgas comprising the remaining gas constituents. In a desorption stage, the C₄ hydrocarbons are released again from the absorbent.

Inert absorbents used in the absorption stage are generally high-boiling nonpolar solvents in which the C₄ hydrocarbon mixture to be removed has a distinctly higher solubility than the remaining gas constituents to be removed. The absorption may be effected by simply passing the product gas stream through the absorbent. However, it may also be effected in columns or in rotary absorbers. It is possible to work in cocurrent, countercurrent or crosscurrent. Examples of suitable absorption columns include tray columns having bubble-cap, centrifugal and/or sieve trays, columns having structured packings, for example sheet metal packings having a specific surface area of from 100 to 1000 m²/m³ such as Mellapak® 250 Y, and randomly packed columns. However, useful absorption columns also include trickle and spray towers, graphite block absorbers, surface absorbers such as thick-film and thin-film absorbers and also rotary columns, plate scrubbers, cross-spray scrubbers and rotary scrubbers.

Suitable absorbents are comparatively nonpolar organic solvents, for example aliphatic C₅- to C₁₈-alkenes, or aromatic hydrocarbons such as the middle oil fractions from paraffin distillation, or ethers having bulky groups, or mixtures of these solvents, to each of which a polar solvent such as dim ethyl 1,2-phthalate may be added. Further suitable absorbents include esters of benzoic acid and phthalic acid with straight-chain C₁-C₈-alkanols, such as n-butyl benzoate, methyl benzoate, ethyl benzoate, dimethyl phthalate, diethyl phthalate, and also heat carrier oils specified, such as biphenyl and diphenyl ether, their chlorine derivatives and also triarylalkenes. A useful absorbent is a mixture of biphenyl and diphenyl ether, preferably in the azeotropic composition, for example the commercially available Diphyl®. Frequently, this solvent mixture comprises dimethyl phthalate in an amount of 0. 1 to 25% by weight. Further suitable absorbents are octanes, nonanes, decanes, undecanes, dodecanes, tridecanes, tetradecanes, pentadecanes, hexadecanes, heptadecanes and octadecanes, or fractions obtained from refinery streams which have the linear alkanes mentioned as main components.

To desorb the C₄ hydrocarbons, the laden absorbent is heated and/or decompressed to a lower pressure. Alternatively, desorption may also be effected by stripping or in a combination of decompression, heating and stripping in one or more process steps. The absorbent regenerated in the desorption stage is recycled into the absorption stage.

In one process variant, the desorption step is carried out by decompressing and/or heating the laden absorbent.

The removal (a1iii) is generally not entirely complete, so that, depending on the type of removal, small amounts or even only traces of the further gas constituents, especially of the low-boiling hydrocarbons, may be present in the C₄ product gas stream.

The C₄ product gas stream obtained after removal of the secondary constituents consists essentially of n-butane, 1-butene, 2-butene and butadiene. In general, the stream comprises from 10 to 80% by volume of n-butane, from 5 to 40% by volume of 1-butene, from 10 to 50% by volume of 2-butene and from 0 to 40% by volume of butadiene. Stream c comprises preferably from 15 to 65% by volume of n-butane, from 10 to 30% by volume of 1-butene, from 15 to 45% by volume of 2-butene and from 1 to 10% by volume of butadiene. In addition, the stream may also comprise small amounts of further gas constituents such as isobutane, isobutene, C₅⁺ hydrocarbons, propane and propene, generally in amounts of from 0 to 10% by volume, preferably from 0 to 5% by volume.

Further preferred embodiments of stage (a1) are described, for example, in WO 2005/042449, WO 2005/063657 and WO 2005/063658.

In a further preferred embodiment of the process according to the invention, n-butane is dehydrogenated in the same reactor simultaneously with the dehydrogenating aromatization of the dimethylhexenes to give o-xylene, or, more preferably, in two reactors connected in series with intermediate feeding.

In this case, an oxygenous stream is also supplied if appropriate to the reactor in which the butane dehydrogenation is performed. The reaction is followed by a separation step in which the C₄ and C₈ fractions are separated from one another, preferably by distillation.

The task of stage (a2) is to convert 1,3-butadiene and 1-butene to 2-butene.

In this stage, the following reactions are therefore implemented simultaneously in the presence of hydrogen:
hydrogenation of 1,3-butadiene to n-butenes,
isomerization of 1-butene to 2-butene according to the thermodynamic equilibrium,
hydrogenation of the acetylenic hydrocarbon traces.

These reactions can be implemented by means of specific catalysts which comprise one or more metals, for example of group 10 of the Periodic Table (Ni, Pd, Pt), deposited on a support. Preference is given to using a catalyst which comprises at least one palladium compound fixed on a heat-resistant mineral support such as alumina. The content of palladium on the support may be between 0.01 and 5% by weight, preferably between 0.05 and 1% by weight. Various pretreatment types known to those skilled in the art may be employed in these catalysts in order to improve the selectivity in the hydrogenation of 1,3-butadiene to butenes. In a preferred embodiment, the catalyst comprises preferably from 0.05 to 10% by weight of sulfur. Particular preference is given to using a catalyst which is formed from palladium deposited on alumina and if appropriate comprises sulfur.

The sulfurization of the catalyst can be effected in situ (in the reaction zone) or preferably before the process is performed. In the latter case, the process employed is., for example, that described in FR-93/09524.

The configuration of the preferably palladium-comprising catalyst is not critical. In general, at least one reactor with a fixed catalyst bed is used, which is operated in descending flow or—for better heat removal—with external circulation. When the content of butadiene in the C₄ stream is relatively high, for example in the case of steamcracker fractions, the reaction is advantageously performed in two reactors connected in series, in order to be able to better control the selectivity of the hydrogenation. The second reactor can then be operated in ascending flow and serves to complete the reaction.

The amount of hydrogen required is calculated from the composition of the stream; preference is given to a slight hydrogen excess.

The operating conditions are preferably selected in such a way that starting materials and products are in the liquid phase. Another advantageous variant is one in which the products are evaporated partially as they leave the reactor, which enables thermal control of the reaction. The reaction is generally performed within a temperature range of from 20 to 200° C., preferably from 50 to 150° C., more preferably from 60 to 150° C. The pressure is generally adjusted to between 0.1 and 5 MPa, preferably between 0.5 and 4 MPa, more preferably between 0.5 and 3 MPa, so that the starting materials are at least partly in liquid form. The LHSV is generally between 0.5 and 10 h⁻¹, preferably between 1 and 6 h⁻¹. The molar ratio of hydrogen to diolefins is generally from 0.5 to 5, preferably from 1 to 3.

If appropriate, traces of excess hydrogen and methane can be removed after the hydroisomerization.

Suitable variants for stage (a2) are described, for example, in WO 2003/035587, EP-A 900 773, EP-A 848 449, EP-A 671 419, U.S. Pat. No. 4,324,938 and WO 01/05734.

Preference is given to a variant described in the latter document.

In this variant, a reactive distillation is performed, which starts from a C₄ stream which comprises butanes, butadiene, n-butenes and if appropriate further components such as isobutene and isobutane. In the reactive distillation, butadiene is hydrogenated to butene and 1-butene is isomerized virtually completely (>95%) to 2-butene. To this end, the C₄ stream is sent through a reactive distillation column which comprises a supported, preferably palladium-containing, catalyst and to which the hydrogen needed to hydrogenate the butadiene is fed simultaneously. In the column, butadiene is hydrogenated to butene and 1-butene is isomerized to 2-butene, which is discharged at the bottom of the column. The lower-boiling components can be drawn off predominantly via the top of the column. Since the 2-butene is removed continually from the reaction zone, isomerization over and above the equilibrium position is possible. Isobutane and isobutene are likewise concentrated and removed via the top, while n-butane in addition to the 2-butene is removed as the bottom product. According to the invention, reactive distillation column refers to a column which additionally comprises a catalyst, so that reaction and distillation are effected simultaneously in the column. The catalyst is preferably used in the form of random packings.

Examples of possible shapes of these random packings are tablets, extrudates, Raschig rings, Pall rings or saddles, and also other such structures, for example spheres, irregular shapes, flat shapes, tubes, spirals, filled into sacks or other constructions (as described, for example, in U.S. Pat. Nos. 4,242,530, 4,443,559, 5,189,001, 5,348,710 and 5,431,890), plated onto grids or screens, or reticulated polymer foams (the cellular structure of the foams has to be sufficiently large so that there is no great pressure drop over the column, or otherwise have to be arranged so as to enable vapor passage, for example in a stacked system or concentration tubes). For example, the catalyst has a structure as disclosed, for example, in U.S. Pat. Nos. 5,730,843, 5,266,546, 4,731,229 and 5,073,236.

The reactive distillation column is generally operated at top temperatures in the range from 20 to 150° C. preferably at from 40° C. to 100° C., and at pressures in the range of from 400 to 1000 kPa gage. In a preferred embodiment, the process is operated under conditions, especially temperature and pressure, under which 2-butene has essentially no contact with the catalyst, while the 1-butene is kept in contact with it. As soon as 1-butene is isomerized to 2-butene, it descends downward out of the catalyst zone in the column and is removed as the bottom product.

The system is preferably operated under reflux. The reflux ratio may generally be from 1 to 100.

Suitable catalysts are described, for example, in EP-A 0 992 284.

The catalyst described there comprises at least one hydrogenation-active metal, preferably from group 8, 9 or 10 of the Periodic Table, more preferably platinum and palladium, on an alumina support, and, in the unused state, exhibits reflections in the X-ray diffractogram which correspond to the following interplanar spacings:

| Interplanar spacing d [10⁻¹⁰ m] | Relative intensity l/lₒ |
|---|---|
| 4.52 | 0.05 to 0.1 |
| 2.85 | 0.35 to 0.45 |
| 2.73 | 0.65 to 0.8 |
| 2.44 | 0.45 to 0.55 |
| 2.31 | 0.35 to 0.45 |
| 2.26 | 0.35 to 0.45 |
| 2.02 | 0.45 to 0.6 |
| 1.91 | 0.3 to 0.4 |
| 1.80 | 0.1 to 0.25 |
| 1.54 | 0.25 to 0.35 |
| 1.51 | 0 to 0.35 |
| 1.49 | 0.2 to 0.3 |
| 1.45 | 0.25 to 0.35 |
| 1.39 | 1 |

Preference is also given to such a catalyst which, in the unused state, exhibits at least one additional reflection in the X-ray diffractogram which corresponds to one of the following interplanar spacings [in 10⁻¹⁰ m]: 3.48, 2.55, 2.38, 2.09, 1.78, 1.74, 1.62, 1.60, 1.57, 1.42, 1.40 and 1.37.

The hydrogenation-active metal is especially preferably palladium and is present in an amount of at least 0.05% by weight and at most 2% by weight based on the total weight of the catalyst.

Preference is further given to a catalyst which, in addition to the hydrogenation-active metal, comprises at least one metal of group 11 of the Periodic Table of the Elements, the metal of group 11 of the Periodic Table of the Elements preferably being copper and/or silver, more preferably silver, and being present in an amount of at least 0.01% by weight and at most 1% by weight based on the total weight of the catalyst.

In the performance of stage (a2), hydrogenation and isomerization can also be effected separately in two steps, i.e., for example, in two reaction zones with different catalysts.

The resulting stream rich in 2-butenes is converted further in process stage (a3).

The invention further provides an apparatus for performing the process according to the invention, as illustrated schematically in FIG. 1.

The apparatus is suitable for processing n-butane-rich streams or streams which already comprise n-butenes or more highly unsaturated C₄ components, for example raffinate II or III. When the starting material is an n-butane-rich stream, the device comprises a zone 101 for dehydrogenating n-butane, this zone further comprising a feed line 106 for an n-butane-containing stream, and feed lines 107, 108 and, if appropriate, 109 for, respectively, steam, recycled n-butane and, if appropriate, an O₂-containing stream, and draw lines 110, 111 and 112 for, respectively, the product stream, H₂O and H₂.

This is followed by a zone 102 for performing the hydrogenation and isomerization. When raffinate II or a comparable C₄ stream is used as the starting material, this zone comprises a feed line 113 for the raffinate II, which is typically combined with the feed line 110 for the product stream of the butane dehydrogenation 101. The zone 102 further comprises a feed line 114 for H₂, and draw lines 115 and if appropriate 116 for, respectively, the 2-butene-rich product stream of the hydrogenation and isomerization and, if the starting material is raffinate II, for i-butane and if appropriate i-butene.

There follows a zone 103 in which the dimerization of 2-butenes to dimethylhexenes (stage (a)) takes place. This zone comprises a feed line 115 for the product stream of the hydrogenation and isomerization zone 102, and draw lines 108, 117 and 118 for, respectively, the recycling of unconverted butane, the dimethylhexene-rich product stream of the dimerization and trimers formed as by-products.

There also follows a zone 104 in which the dehydrogenating aromatization of the dimethylhexenes to o-xylene takes place. This zone 4 comprises feed lines 117, 119 and 120 for, respectively, the product stream of the dimerization, steam and unconverted dimers, and draw lines 121, 122 and 123 for, respectively, the o-xylene-containing product stream, H₂ and offgas, and H₂O.

Finally, there follows a zone 105 for distillative purification of the resulting o-xylenes. This zone comprises a feed line 121 for the product stream of the dehydrogenating aromatization, and draw lines 120, 124 and 125 for, respectively, unconverted dimethylhexenes, further high and low boilers, and the desired o-xylene.

The invention further provides apparatus for performing the process according to the invention, in which dehydrogenating aromatization and butane dehydrogenation are performed in one reactor or in two reactors connected in series with intermediate feeding. Such apparatus is shown schematically in FIGS. 2 and 3.

The apparatus according to FIG. 2 or 3 is suitable for the processing of n-butane-rich streams or of streams which already comprise n-butenes or more highly unsaturated C₄ components, for example raffinate II or III.

When the starting material is an n-butane-rich stream, the apparatus according to FIG. 2 comprises a zone 201 for dehydrogenating n-butane, which simultaneously serves for the dehydrogenating aromatization (stage (b)). Stage 201 comprises feeds 206, 207, 208, 209 and if appropriate 210 for, respectively, the n-butane-containing starting material, the product stream of the dimerization zone 204, recycled butane, steam and if appropriate an O₂-containing stream, and draw lines 211, 212 and 213 for, respectively, the product stream which is composed of an unsaturated C₄ stream and an o-xylene-rich stream, hydrogen and H₂O.

This is followed by a zone 202 for separating the C₄ and C₈ streams obtained in zone 201, comprising a feed line 211 and draw lines 214 and 215 for the C₄ and C₈ stream respectively.

The C₄ stream is followed by a zone 203 for performing the hydrogenation and isomerization, which has feed lines 214 and 216 for the C₄ stream and hydrogen respectively. When raffinate II or raffinate III is employed instead of an n-butane-rich starting stream, zone 203 also comprises a feed line 217 for raffinate II or raffinate III (if appropriate combined with the feed line 214 for the C₄ stream). Zone 203 further comprises draw lines 218 and if appropriate 219 for, respectively, the product stream rich in 2-butenes and, if appropriate, for i-butane and/or i-butene.

There follows a zone 204 for performing the dimerization, which comprises a feed line 218 for the product stream of the hydrogenation and isomerization, and draw lines 207, 208 and 220 for, respectively, the C₈ product stream of the dimerization, unused butane and trimers formed as by-products. Draw lines 207 and 208 lead into zone 201, the C₈ product stream (draw line 207), if appropriate, being combined with a feed line 221 of unconverted dimers from the distillation zone 205.

After passing through zones 201 and 202 for dehydrogenating aromatization and removal of C₄ products, the o-xylene-rich C₈ stream from zone 202 is passed via a draw line 215 into a zone 205 where o-xylene is purified by distillation. In addition to the feed 215, this zone comprises draw lines 221, 222 and 223 for, respectively, unconverted dimers, high and low boilers, and the desired product, o-xylene.

The apparatus according to FIG. 3 comprises a zone 301 for dehydrogenating n-butane, this zone further comprising a feed line 307 for an n-butane-containing stream and feed lines 308, 309 and if appropriate 310 for, respectively, steam, recycled n-butane and, if appropriate, an O₂-containing stream, and a draw line 311 for the product stream.

This is followed by a zone 302 for dehydrogenating aromatization. The zone 302 comprises feeds 311, 312 and if appropriate 313 for, respectively, the product stream of the butane dehydrogenation, the product stream of the dimerization and, if appropriate, steam and draw lines 314, 315 and 316 for, respectively, the product stream which is composed of an unsaturated C₄ stream and an o-xylene-rich stream, hydrogen and H₂O.

This is followed by a zone 303 for separating the C₄ and C₈ streams, comprising a feed line 314 and draw lines 317 and 318 for the C₄ and C₈ stream respectively.

The C₄ stream is followed by a zone 304 for performing the hydrogenation and isomerization, which has feed lines 317 and 319 for the C₄ stream and hydrogen respectively. When raffinate II or raffinate III is employed instead of an n-butane-rich starting stream, the zone 304 also comprises a feed line 320 for raffinate II or raffinate III (if appropriate combined with the feed line 317 for the C₄ stream). Zone 304 further comprises draw lines 321 and if appropriate 322 for, respectively, the product stream rich in 2-butenes and, if appropriate, for i-butane and/or i-butene.

There follows a zone 305 for performing the dimerization, which comprises a feed line 321 for the product stream of the hydrogenation and isomerization, and draw lines 309, 312 and 323 for, respectively, the C₈ product stream of the dimerization, unconverted butane and trimers formed as by-products. Draw lines 309 and 312 lead into zone 301 and 302, the C₈ product stream (draw line 312) being combined if appropriate with a feed line 324 of unconverted dimers from the distillation zone 306.

After passing through zones 302 and 303 for dehydrogenating aromatization and removal of C₄ products, the o-xylene-rich C₈ stream from zone 302 is passed through a draw line 318 into a zone 306 where o-xylene is purified by distillation. In addition to the feed 318, this zone comprises draw lines 324, 325 and 326 for, respectively, unconverted dimers, high and low boilers, and the desired product, o-xylene.

The invention is illustrated in details by the examples.

### EXAMPLES

A Dimerization of 2-Butene/Butane Mixtures

Catalyst Preparation

### Example 1

400 g of an alumina support (1.5×1.5 mm tablets D10-21, BASF) were impregnated with a solution of 81 g of 96% H₂SO₄ in water at room temperature to water absorption. After drying at 120° C. in air, calcination was finally effected at 500° C. in a rotary tube with passage of 300 l/h of air for 2 h.

### Example 2

The procedure is as in Example 1) except that 400 g of a titanium oxide support DT51 (4 mm of extrudates prepared from 7.5 kg of DT51 (from Thann et Mulhouse), which have been compacted with 2.5 kg of water and 420 g of 85% formic acid in a pan grinder for 1 h and shaped by means of an extruder and calcined at 500° C.) are used.

### Examples 3 to 5

The procedure is as described under 1) except that 400 g of a silicon aluminum oxide Siral 5, 10 or 40 (4 mm extrudates prepared from 500 g of Siral (Condea), which had been compacted with from 380 to 400 ml of H₂O and 15 ml of concentrated HNO₃ in a kneader for 1 h and, after addition of 10 g of methylcellulose (Walocel, from Wolf) and 5 g of PEO (polyethylene oxide), have been shaped by means of an extrudate press and calcined at 500° C.) are used.

### Example 6

400 g of a titanium dioxide support DT51 (see Example 2) were impregnated with a solution of 80 g of H₄SiW₁₂O₄₀ in water at room temperature for water absorption. After drying at 120° C. in air, calcination was finally effected at 350° C. in a rotary tube while passing through 300 l/h of air for 2 h.

### Example 7

400 g of a silicon aluminum oxide support (4 mm Siral 40 extrudates, see Example 3) were impregnated with a solution of 100.4 g of CoSO₄.7H₂O in 800 ml of water at room temperature. After being left to stand for 16 hours, the supernatant solution was removed and then dried at 120° C. in air, and calcined at 500° C. in a rotary tube while passing through 300 l/h of air for 2 h.

### Example 8

400 g of a silicon aluminum oxide support (4 mm Siral 40 extrudates, see Example 3) were impregnated with a solution of 104.8 g of FeSO₄.7H₂O in 800 ml of water at room temperature. After being left to stand for 16 hours, the supernatant solution was removed and then dried at 120° C. in air, and calcined at 500° C. in a rotary tube while passing through 300 l/h of air for 2 h.

### Example 9

The feedstock used was 99.5% 2-butene (from Gerling-Holz or Linde). The reaction was performed in a tubular reactor of nominal width 10 mm at temperatures of from 25 to 30° C. and a pressure of 20 bar. The reactor was charged with 10 g of catalyst and, before startup, activated at ambient pressure at 200° C. in an N2 stream (10 l/h) over 24 h. After cooling to approx. 25° C., the supply was switched to 2-butene with an increase in the reaction pressure to 20 bar. 10 g of 2-butene per hour were supplied and removed. In addition, 1000 g of reaction mixture per h were circulated. The analysis was effected by means of online gas chromatography. The table below shows a summary of the results obtained after from 48 to 72 h of run time.

| | | | C₈ isomer distribution | | | | | |
|---|---|---|---|---|---|---|---|---|
| Catalyst | | | 3,4 Me₂-C₆ | | 2,4 Me₂C₆ | | | |
| according | Conversion | C₈ | and | | and | | 3,3 | Other |
| to Example | (2-butene) | selectivity | 2,3 Me₂-C₆ | Me-C₇ | 2,5 Me₂-C₆ | EtMe-C₅ | Me-C₆ | C₈ |
| 1 | 24 | 86 | 94.0 | 0.9 | 1.2 | 1.7 | 1.1 | 1.1 |
| 2 | 35 | 85 | 93.8 | 1.0 | 1.3 | 1.7 | 1.1 | 1.1 |
| 3 | 25 | 89 | 95.5 | 0.5 | 1.1 | 1.4 | 0.9 | 0.6 |
| 4 | 25 | 88 | 94.5 | 0.7 | 1.3 | 1.5 | 1.3 | 0.7 |
| 5 | 30 | 89 | 94.4 | 1.2 | 0.9 | 1.9 | 0.9 | 0.7 |
| 6 | 20 | 89 | 94.2 | 1.0 | 1.0 | 1.8 | 1.2 | 0.9 |
| 7 | 8 | 89 | 90.0 | 2.1 | 2.8 | 3.0 | 1.1 | 1.0 |
| 8 | 10 | 88 | 92.8 | 1.7 | 1.6 | 2.2 | 1.0 | 0.7 |

B Dehydrogenation Aromatization of Dimethylhexenes

### Example 10

Catalyst Preparation

The catalyst (10) was prepared according to DE 199 37 107, Example 4.

Reaction Procedure

The dehydrogenating aromatization was performed in an electrically heated tubular reactor. About 24 g of catalyst were installed into the reactor and activated with hydrogen at 400° C. for 1 h. The feedstock used was dimerization product from Example 3, whose C₈ fraction comprised approx. 95% by weight of o-xylene precursors, mainly 3,4- and 2,3-dimethyl-2-hexene (3,4- and 2,3-DMH) respectively. The table shows results of the dehydrogenating aromatization at oven temperature 450° C. 1 bar(abs), WHSV (total organics) 0.6 h⁻¹, H₂O/DMH 16/1 mol/mol and H₂/DMH 3/1 mol/mol after one hour of run time.

### Example 11

Catalyst Preparation

100 g of La—Ce—ZrO₂ support (MEL Chemicals, 1.5-2.5 mm spall, solvent absorption 0.37 cm³/g) were impregnated with 37 ml of KOH solution (concentration 5%) according to the water absorption. After a contact time of 2 h, the impregnated support was dried at 120° C. for 30 min. In the second impregnation step, 33.23 g of tetraamineplatinum(II) nitrate solution (3.3% Pt content) were made up to 37 ml of overall solution with deionized water and applied to the support. After a contact time of 2 h, the impregnated support was dried at 120° C. for 30 min. Thereafter, in the third impregnation step, 5.002 g of SnCl₂.2H₂O and 17.089 g of La(NO₃)₃.6H₂0, dissolved in 35 ml of applied. After a contact time of 2 h, the catalyst was dried at 120° C. for 30 min and then calcined at 560° C. for 3 h. The catalyst (11) comprised 1% Pt, 0.4% K, 2.4% Sn, 5% La and 91.2% support.

Reaction Procedure

The procedure was analogous to Example 10. The table shows results of the dehydrogenating aromatization at oven temperature 525° C., 3 bar(abs), WHSV (total organics) 1.6 h⁻¹, H₂O/DMH 22/1 mol/mol and H₂/DMH 1.7/1 mol/mol after one hour of run time.

### Example 12

Catalyst Preparation

The catalyst (12) was prepared analogously to Example 11. The catalyst comprises 0.6% Pt, 0.3% Ga, 2.4% Sn, 5% La and 91.7% support. Pt was applied in the first impregnation step, and Sn, La and Ga (in the form of dissolved Ga(NO₃)₃.9H₂O) correspondingly in the second.

Reaction Procedure

The procedure was analogous to Example 10. The table shows results of the dehydrogenation aromatization at oven temperature 500° C., 1 bar(abs), WHSV (total organics) 0.6 h⁻¹, H₂O/DMH 16/1 mol/mol and H₂/DMH 16/1 mol/mol after one hour of run time.

| | | | o-Xylene/(m- | o-Xylene | |
|---|---|---|---|---|---|
| Catalyst | | Xylene | xylene + p- | space-time | |
| according | DMH conversion | selectivity | xylene) ratio | yield | C8 selectivity |
| to Example | [%] | [%] | [—] | [g_{o-xylene}/ml_{cat}/h] | [%] |
| 10 | 53 | 46 | 19 | 0.16 | 99 |
| 11 | 58 | 71 | 26 | 0.83 | 97 |
| 12 | 79 | 80 | 19 | 0.42 | 98 |

C Dehydrogenation of n-Butane/Isomerization to 2-Butenes

### Example 13

Catalyst Preparation

The catalyst was prepared according to DE 199 37 107, Example 4.

Reaction Procedure

The dehydrogenation of n-butane was performed in an electrically heated tubular reactor. About 24 g of catalyst were installed into the reactor and activated with hydrogen at 500° C. for 1 h. The use mixture had the following composition: n-C₄H₁₀:H₂:H₂O:N₂:O₂=8:1:4:0.9:0.2 mol/mol. The table which follows shows results of the n-butane dehydrogenation at 550° C. 1.5 bara, GHSV (n-butane) 650 h⁻¹:

| | | | |
|---|---|---|---|
| n-Butane | 2-Butene | 1-Butene | Butadiene |
| conversion | selectivity | selectivity | selectivity |
| [%] | [%] | [%] | [%] |
| after | after | after | after |

| 1 h | 10 h | 1 h | 10 h | 1 h | 10 h | 1 h | 10 h |
|---|---|---|---|---|---|---|---|
| 43 | 42 | 63 | 62 | 30 | 30 | 3 | 4 |

### Example 14

### Hydrogenation and Isomerization of C₄ Mixture from a Butane Dehydrogenation

The reaction is performed in reactive distillation mode in order to be able to shift the proportion of 2-butenes over and above the equilibrium position. To this end, the C₄ mixture from the butane dehydrogenation is conducted into a column having a diameter of 50 mm in which a catalyst bed of Pd on Al₂O₃ (1.5 mm extrudates) in multichannel packings has been installed and which has about 40 theoretical plates. The feed is conducted into the column directly above the catalyst bed. Above (1 to 5 plates) and below (35 to 40 plates) the catalyst zone, distillation packings are installed.

The column is operated at a pressure of about 8 bar absolute, i.e. a temperature of about 70° C. is present in the reaction zone. The feed rate is 500 g/h, the mass of catalyst installed 500 g, the hydrogen flow rate from about 1 to 3 l (STP)/h.

The column is operated with complete reflux. Small amounts of C₄ in the tops are lost only via the offgas. At the bottom, a mixture which consists predominantly of 2-butenes and n-butane is drawn off.

The following result is obtained:

| Components | Feed stream % | Bottom effluent % | Top effluent % |
|---|---|---|---|
| n-Butane | 57.3 | 57.3 | 65.6 |
| 1-Butene | 13.0 | 0.4 | 0.6 |
| cis-2-Butene | 12.1 | 19.2 | 6.6 |
| trans-2-Butene | 16.0 | 23.1 | 27.2 |
| 1,3-Butadiene | 1.6 | 0 | 100 ppm |
| | g/h | g/h | g/h |
| Amount | 500 | 490 | 10 |

### Example 15

### Isomerization of Raffinate II

The reaction is performed in reactive distillation mode in order to be able to shift the content of 2-butenes over and above the equilibrium position. To this end, raffinate II is conducted into a column having a diameter of 50 mm, in which a catalyst bed of Pd on Al₂O₃ (1.5 mm extrudates) has been installed in multichannel packings and which has about 40 theoretical plates. The catalyst is installed in the uppermost part of the column. The feed is conducted directly into the catalyst bed. Below the catalyst zone, distillation packings are installed. Compared to the feed from Example 14, raffinate II additionally comprises small amounts of isobutane and isobutene which are drawn off via the top.

The column is operated at a pressure of approx. 8.4 bar absolute, i.e. a temperature of approx. 65° C. is present in the reaction zone. The feed rate is 500 g/h, the mass of catalyst installed 500 g, the hydrogen flow rate from approx. 1 to 3 l (STP)/h.

The column is operated with high reflux ratio and with low top draw. At the bottom, a mixture which consists predominantly of 2-butenes and n-butane is drawn off.

The following result is obtained:

| Components | Feed stream % | Bottom effluent % | Top effluent % |
|---|---|---|---|
| Isobutane | 3.9 | 0 | 25.4 |
| Isobutene | 2.7 | 0.1 | 17.2 |
| 1-Butene | 49.7 | 0.6 | 25.6 |
| cis-2-Butene | 10.2 | 32.8 | 6.3 |
| trans-2-Butene | 21.4 | 52.5 | 18.2 |
| n-Butane | 12.0 | 13.9 | 7.2 |
| | g/h | g/h | g/h |
| Amount | 500 | 414 | 86 |

1.-11. (canceled)

12. A process for preparing o-xylene, comprising the steps of
a) dimerizing 2-butenes to 3,4- and/or 2,3-dimethylhexenes and
b) aromatizing the 3,4- and/or 2,3-dimethylhexenes under dehydrogenating conditions to give o-xylene.

13. The process according to claim 12, comprising the steps of
(a1) dehydrogenating butane to give an n-butane/n-butenes/n-butadiene mixture,
(a2) selectively hydrogenating (a1) under isomerizing conditions to give a mixture comprising n-butane/2-butenes,
(a3) dimerizing the 2-butenes from (a2) to give a C₈₊ product mixture whose C₈ fraction comprises predominantly 3,4- and 2,3-dimethylhexenes and
(b) aromatizing the C₈ fraction obtained from (a3) under dehydrogenating conditions to give o-xylene.

14. The process according to claim 13, wherein the butane dehydrogenation (a1) and the dehydrogenating aromatization (b) are performed in separate reactors.

15. The process according to claim 13, wherein the butane dehydrogenation (a1) and the dehydrogenating aromatization (b) are performed in the same reactor.

16. The process according to claim 13, wherein the butane dehydrogenation (a1) and the dehydrogenating aromatization (b) are performed in two reactors connected in series.

17. The process according to claim 13, wherein stream (a2) comprises less than 5% by weight of 1-butene based on the overall butenes.

18. The process according to claim 12, wherein the C₈ fraction of stream a) consists to an extent of more than 90% by weight of 3,4- and 2,3-dimethylhexenes.

19. The process according to claim 13, wherein the C₈ fraction of stream a) consists to an extent of more than 90% by weight of 3,4- and 2,3-dimethylhexenes.

20. The process according to claim 12, wherein the o-xylene stream obtained in (b) has an index [% by weight of o-xylene/(% by weight of m- and p-xylene)] of >50.

21. The process according to claim 13, wherein the o-xylene stream obtained in (b) has an index [% by weight of o-xylene/(% by weight of m- and p-xylene)] of >50.

22. An apparatus for performing the process according to claim 14, comprising
a zone (101) for dehydrogenating n-butane, this zone further comprising a feed line (106) for an n-butane-containing stream and feed lines (107), (108) and, if appropriate, (109) for, respectively, steam, hydrogen, recycled n-butane and, if appropriate, an O₂-containing stream, and draw lines (110), (111) and (112) for, respectively, the product stream, offgas, H₂O and H₂;
a zone (102) for performing the hydrogenation and isomerization, comprising a feed line (113) for raffinate II or raffinate III, which is combined with the feed line (110) for the product stream of the butane dehydrogenation (101), a feed line (114) for H₂ and draw lines (115) and if appropriate (116) for, respectively, the 2-butene-rich product stream of the hydrogenation and isomerization and, if the starting material is raffinate II or raffinate III, for i-butane and if appropriate i-butene;
a zone (103) in which the dimerization of 2-butenes to dimethylhexenes (stage (a)) takes place, comprising a feed line (115) for the product stream of the hydrogenation and isomerization zone (102) and draw lines (108), (117) and (118) for, respectively, the recycling of unconverted butane, the dimethylhexene-rich product stream of the dimerization and trimers formed as by-products;
a zone (104) in which the dehydrogenating aromatization of the dimethylhexenes to o-xylene takes place, comprising feed lines (117), (119) and (120) for, respectively, the product stream of the dimerization, steam, if appropriate hydrogen and an O₂-containing stream, and unconverted dimers, and draw lines (121), (122) and (123) for, respectively, the o-xylene-containing product stream, H₂ and offgas, and H₂O;
and a zone (105) for distillative purification of the resulting o-xylenes, comprising a feed line (121) for the product stream of the dehydrogenating aromatization and draw lines (120), (124) and (125) for, respectively, unconverted dimethylhexenes, further high and low boilers and o-xylene.

23. An apparatus for performing the process according to claim 15, comprising
a zone (201) for dehydrogenating n-butane which simultaneously serves for the dehydrogenating aromatization (stage (b)), comprising feeds (206), (207), (208), (209) and if appropriate (210) for, respectively, the n-butane-containing starting material, the product stream of the dimerization zone (204), recycled butane, steam and, if appropriate, an O₂-containing stream and hydrogen, and draw lines (211), (212) and (213) for, respectively, the product stream which is composed of an unsaturated C₄ stream and an o-xylene-rich stream, hydrogen and H₂O;
a zone (202) for separating the C₄ and C₈ streams obtained in zone (201 ), comprising a feed line (211) and draw lines (214) and (215) for the C₄ and C₈ stream respectively;
a zone (203) for performing the hydrogenation and isomerization which has feed lines (214) and (216) for the C₄ stream and hydrogen respectively, comprising a feed line (217) for raffinate II or raffinate III (if appropriate combined with the feed line (214) for the C₄ stream), and also draw lines (218) and if appropriate (219) for, respectively, the product stream rich in 2-butenes and, if appropriate, for i-butane and/or i-butene;
a zone (204) for performing the dimerization which comprises a feed line (218) for the product stream of the hydrogenation and isomerization, and draw lines (207), (208) and (220) for, respectively, the C₈ product stream of the dimerization, unused butane and trimers formed as by-products, draw lines (207) and (208) into the zone (201), the C₈ product stream (draw line 207), if appropriate, being combined with a feed line (221) of unconverted dimers from the distillation zone (205); and
a zone (205), where o-xylene is purified by distillation, comprising a feed (215) and draw lines (221), (222) and (223) for, respectively, unconverted dimers, high and low boilers and o-xylene.

24. An apparatus for performing the process according to claim 16, comprising
a zone (301) for dehydrogenating n-butane, this zone further comprising a feed line (307) for an n-butane-containing stream and feed lines (308), (309) and if appropriate (310) for, respectively, steam, recycled n-butane and, if appropriate, hydrogen and an O₂-containing stream, and a draw line (311) for the product stream;
a zone (302) for dehydrogenating aromatization, comprising feeds (311), (312) and if appropriate (313) for, respectively, the product stream of the butane dehydrogenation, the product stream of the dimerization and, if appropriate, hydrogen, an O₂-containing stream and steam, and draw lines (314), (315) and (316) for, respectively, the product stream which is composed of an unsaturated C₄ stream and an o-xylene-rich stream, hydrogen and H₂O;
a zone (303) for separating the C₄ and C₈ streams, comprising a feed line (314) and feed lines (317) and (318) for the C₄ and C₈ stream respectively;
a zone (304) for performing the hydrogenation and isomerization which has feed lines (317) and (319) for the C₄ stream and hydrogen respectively, comprising a feed line (320) for raffinate II or raffinate III (if appropriate combined with the feed line (317) for the C₄ stream), and also draw lines (321) and if appropriate (322) for, respectively, the product stream rich in 2-butenes and, if appropriate, for i-butane and/or i-butene;
a zone 305 for performing the dimerization which comprises a feed line (321) for the product stream of the hydrogenation and isomerization, and draw lines (309), (312) and (323) for, respectively, the C₈ product stream of the dimerization, unused butane and trimers formed as by-products, and draw lines (309) and (312) into the zone (301) and (302), the C₈ product stream (draw line 312) being combined if appropriate with a feed line (324) of unconverted dimers from the distillation zone (306); and
a zone (306), where o-xylene is purified by distillation, comprising, in addition to the feed (318), draw lines (324), (325) and (326) for, respectively, unconverted dimers, high and low boilers and the desired product, o-xylene.
